(19) United States
(12) Patent Application Publication
(10) Pub. No.: US 2012/0277745 A1
(43) Pub. Date: Nov. 1, 2012

(54) SYSTEMS AND METHODS FOR POSITIONING A FOOT IN ANKLE ARTHRODESIS

(76) Inventor: Emmanuel LIZEE, Saint Ismier (FR)

(21) Appl. No.: 13/278,543

(22) Filed: Oct. 21, 2011

Related U.S. Application Data
(63) Continuation-in-part of application No. PCT/EP2010/055218, filed on Apr. 20, 2010.
(60) Provisional application No. 61/171,344, filed on Apr. 21, 2009.

(30) Foreign Application Priority Data
Sep. 23, 2011 (FR) 1158510

(51) Int. Cl. A61B 17/00; G01B 5/24

(52) U.S. Cl. 606/59; 33/512

(57) ABSTRACT

A positioning guide for ankle arthrodesis according to some embodiments of the present invention includes a clamping device configured to be fixedly held to the foot, the clamping device defining an implantation axis, an alignment device configured to be removably coupled with the clamping device, the alignment device having first and second angle indicators, wherein, when the alignment device is coupled to the clamping device, the first angle indicator is configured to visually indicate a substantial alignment of implantation axis with tibial when the first angle indicator is aligned with the tibial axis and the foot is placed in a first position, and the second angle indicator is configured to visually indicate an arthrodesis angle formed between implantation axis and tibial axis when the second angle indicator is aligned with the tibial axis and the foot is placed in a second position.

### CROSS-REFERENCE TO RELATED APPLICATIONS

This application is a continuation-in-part of Patent Cooperation Treaty Application Number PCT/EP2010/055218, filed on Apr. 20, 2010, which claims the benefit of U.S. Provisional Patent Application Ser. No. 61/171,344, filed on Apr. 21, 2009; this application also claims foreign priority to French patent application no. FR1158510, filed on Sep. 23, 2011, all of which are incorporated by reference herein in their entireties for all purposes.

### TECHNICAL FIELD

Embodiments of the present invention relate generally to bone positioning, and more specifically to systems and methods for ankle positioning for arthrodesis.

### BACKGROUND

The orientation and positioning of the ankle during arthrodesis is often an important aspect of the procedure, and will also determine the ankle's position after surgery. Surgeons who perform ankle arthrodesis currently often orient and position the ankle using their unaided visual judgment and/or trial-and-error positioning methods. After a desired ankle position is achieved, surgeons also often experience difficulty in maintaining the desired position during drilling and other aspects of the procedure. Such basic positioning methods often increase the time of the procedure, lead to inconsistency in different procedures, and may result in less-than-optimal positioning.

### SUMMARY

Some arthrodesis procedures require the implantation of a bent or curved nail or support bar through the calcaneus, the talus, and into the tibia. For example, the implantation nail may include a valgus angle of approximately six degrees. However, although the implantation nail is bent, the hole drilled through the calcaneus, talus, and tibia must be relatively straight. Systems and methods according to embodiments of the present invention permit the ankle to be positioned, rotated between the arthrodesis position and the drilling position with a higher degree of precision, and secured in the drilling position for easier and safer drilling.

A foot positioning device according to embodiments of the present invention includes a clamping device and an alignment device. The clamping device includes an upper arm placed above the talus and a screw arm placed under the calcaneus. The screw arm is tightened until the clamping device is held to the ankle by a compression of the talus and calcaneus between the upper arm and the screw arm, according to embodiments of the present invention. Once attached in this manner, the clamping device moves with the ankle as the ankle is rotated with respect to the tibia. The alignment device includes one or more screws or pegs that slide within one or more slots on the clamping device, as well as alignment rods extending from the alignment device. The alignment device is placed onto the clamping device, and a desired foot position is found at least in part by aligning a bent valgus anterior rod and a lateral rod of the alignment device with the mechanical axis of the tibia. The angle of the bent valgus anterior rod corresponds substantially with the valgus angle of the selected arthrodesis implant nail, according to embodiments of the present invention.

The alignment device is attached (e.g. by a compression screw) to the clamping device, and then the ankle is rotated through an angle to align an unbent valgus anterior rod and the lateral rod of the alignment device with the mechanical axis of the tibia, to place the ankle in the drilling position. Once in the drilling position, one or more pins may be inserted through apertures in the alignment device and into the tibia, to secure the alignment device to the tibia. In addition, one or more pins may be inserted through an annular opening in the screw arm, through the calcaneus, the talus, and into the tibia, to further secure the alignment device to the foot and to prevent undesired rotation of the ankle during subsequent drilling and/or preparation steps.

A positioning guide for ankle arthrodesis with respect to a foot and a tibia, the tibia having a tibial axis, according to embodiments of the present invention includes a clamping device configured to be fixedly held to the foot, the clamping device defining an implantation axis through the foot, an alignment device configured to be removably coupled with the clamping device, the alignment device comprising a first angle indicator and a second angle indicator, wherein, when the alignment device is coupled to the clamping device, the first angle indicator is configured to visually indicate a substantial alignment of the implantation axis with the tibial axis in at least one plane when the first angle indicator is aligned with the tibial axis in the at least one plane and the foot is placed in a first position, and the second angle indicator is configured to visually indicate an arthrodesis angle formed between the implantation axis and the tibial axis in the at least one plane when the second angle indicator is aligned with the tibial axis in the at least one plane and the foot is placed in a second position.

According to some embodiments of the present invention, the foot includes a talus and a calcaneus, the clamping device includes a talus fork configured for placement on a top of a dome of the talus and a locking screw assembly configured for placement on a bottom of the calcaneus, and the locking screw assembly is configured to be tightened to compress the talus and the calcaneus to fixedly hold the clamping device to the talus and the calcaneus. The clamping device may include a handle which, in turn, includes a hole positioned along the implantation axis and configured to receive the locking screw assembly, and a lateral slot formed between the hole and an exterior of the handle, the lateral slot configured to receive an orthopedic guide pin to permit sliding of the orthopedic guide pin from the exterior to within the hole. The clamping device may further include a first upper support bar, the first supper support bar including the talus fork, the positioning guide further including a second upper support bar with a different shape than the first upper support bar, such that the first and second upper support bars are interchangeable with respect to the clamping device to permit the clamping device to be applied to the foot about different angular orientations with respect to the implantation axis. The locking screw assembly may include an annular opening that defines the implantation axis, and the implantation axis may pass through the talus fork, according to embodiments of the present invention.

According to some embodiments of the present invention, the alignment device further includes a third angle indicator, wherein, when the alignment device is coupled to the clamping device, the third angle indicator is configured for visual alignment with the tibial axis both when the foot is in the first position and when the foot is in the second position. In some cases, the at least one plane is a coronal plane, and at least a portion of the third angle indicator is configured to remain in the coronal plane when the foot is in the first and second positions. In other cases, the at least one plane is a first plane, and the third angle indicator extends substantially along a second plane that is orthogonal to the first plane and includes the implantation axis. According to embodiments of the present invention, the first, second, and third angle indicators are configured such that, when the clamping device is fixedly held to the foot and the alignment device is coupled to the clamping device, the first and second angle indicators are positioned anterior to the tibia and the third angle indicator is positioned lateral to the tibia. The first, second, and third angle indicators may be rotatable about the implantation axis with respect to the clamping device before the alignment device is removably coupled with the clamping device. In some cases, the first, second, and/or third angle indicators are rods.

According to some embodiments of the present invention, the alignment device includes a protractor with angle markings, and the angle markings include the first and second angle indicators. The alignment device may further include a base and a protractor rod connecting the protractor with the base, wherein the protractor rod is configured to visually indicate a substantial alignment of the implantation axis with the tibial axis in the at least one plane when the protractor rod is aligned with the tibial axis in the at least one plane and the foot is placed in the first position. The protractor may include the angle markings both laterally and medially of the first angle indicator to permit use of the alignment device with both a left foot arthrodesis and a right foot arthrodesis, according to embodiments of the present invention.

A method for measuring an arthrodesis angle with respect to a foot and a tibia, the tibia having a tibial axis, according to embodiments of the present invention includes placing a clamping device fixedly onto a foot, the clamping device defining an implantation axis through the foot, coupling an alignment device with the clamping device, the alignment device comprising a first angle indicator and a second angle indicator, placing the foot in a first position in which the first angle indicator is aligned with the tibial axis to visually indicate substantial alignment of the implantation axis with the tibial axis, and placing the foot in a second position in which the second angle indicator is aligned with the tibial axis to visually indicate an arthrodesis angle formed between the implantation axis and the tibial axis.

According to some embodiments of the present invention, the alignment device further includes a protractor with angle markings, wherein the angle markings include the first angle indicator and the second angle indicator, such that placing the foot in the first position includes aligning an index marking of the angle markings with the tibial axis, and placing the foot in the second position includes placing the foot in a desired arthrodesis position and observing which of the angle markings is aligned with the tibial axis. According to some embodiments of the present invention, the arthrodesis angle may be determined by determining an angular difference between the index marking and the marking aligned with the tibial axis in the second position. The alignment device may further include a third angle indicator, wherein placing the foot in the first position includes aligning the third angle indicator (e.g. laterally) with the tibial axis, and wherein placing the foot in the second position comprises aligning the third angle indicator (e.g. laterally) with the tibial axis. According to some embodiments of the present invention, the first and second angle indicators are anterior rods, and the alignment device further includes a third angle indicator that is a lateral rod, wherein placing the foot in the first position includes aligning the lateral rod laterally with the tibial axis, and wherein placing the foot in the second position includes aligning the lateral rod laterally with the tibial axis.

Such methods may further include rotating the alignment device about the implantation axis before coupling the alignment device to the clamping device, to position the first and second angle indicators anteriorly and substantially along a coronal plane with respect to the foot, according to embodiments of the present invention. The clamping device may include a talus fork and a locking screw assembly, and placing the clamping device fixedly onto the foot includes placing the talus fork on a top of a dome of a talus of the foot and placing the locking screw assembly on a bottom of a calcaneus of the foot, the method further including tightening the locking screw assembly to compress the calcaneus and the talus to fixedly hold the clamping device to the talus and the calcaneus, according to embodiments of the present invention. According to some embodiments of the present invention, a rod (or screw or other fixation element) may be placed into the calcaneus, in addition to or instead of using the locking assembly, to hold the clamping device to the foot.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a front perspective view of a clamping device, according to embodiments of the present invention.

FIG. 2 illustrates a front perspective view of an alignment device, according to embodiments of the present invention.

FIG. 3 illustrates a front perspective view of the clamping device of FIG. 1 being applied to a foot, according to embodiments of the present invention.

FIG. 4 illustrates a front perspective view of the alignment device of FIG. 2 being applied to the clamping device, according to embodiments of the present invention.

FIG. 5 illustrates a front perspective view of a clamping device and an alignment device, according to embodiments of the present invention.

FIG. 6 illustrates a front elevation view of a valgus anterior alignment rod substantially aligned with a mechanical axis of a tibia, according to embodiments of the present invention.

FIG. 7 illustrates a side elevation view of another alignment rod substantially aligned with a mechanical axis of a tibia, according to embodiments of the present invention.

FIG. 8 illustrates a front elevation view of drilling position alignment rod substantially aligned with a mechanical axis of a tibia, according to embodiments of the present invention.

FIG. 9 illustrates a front perspective view of fixation pins inserted through the alignment device and the clamping device into the tibia, the calcaneus, and the talus, according to embodiments of the present invention.

FIG. 10 illustrates a front perspective view of an alternative embodiment of a clamping device, according to embodiments of the present invention.

FIG. 11 depicts a flow chart illustrating a method for using an alignment device and clamping device, according to embodiments of the present invention.

FIG. 12 illustrates a perspective view of a positioning guide, according to embodiments of the present invention.

FIG. 13 illustrates another perspective view of the positioning guide of FIG. 12, according to embodiments of the present invention.

FIG. 14 illustrates a perspective view of a clamping device applied to an ankle, according to embodiments of the present invention.

FIG. 15 illustrates a top plan view of the clamping device of FIG. 14 applied to foot bones, according to embodiments of the present invention.

FIG. 16 illustrates the clamping device of FIG. 14 with an alignment device coupled thereto, according to embodiments of the present invention.

FIG. 17 illustrates a front elevation view of the positioning guide of FIGS. 12 and 16 applied to a foot which is in an arthrodesis position, according to embodiments of the present invention.

FIG. 18 illustrates a front elevation view of the positioning guide of FIGS. 12 and 16 applied to a foot which is in an implantation position, according to embodiments of the present invention.

FIG. 19 illustrates a set of two alternative upper support bars for use with a clamping device, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present invention permit surgeons to more precisely move the subject's foot between a desired arthrodesis position and an alternative position in which drilling or other preparatory steps are performed.

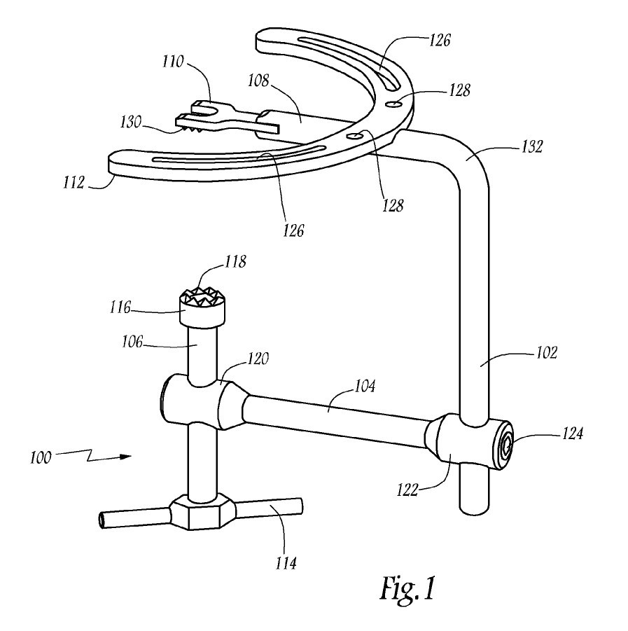

FIG. 1 illustrates a front perspective view of a clamping device 100, according to embodiments of the present invention. Clamping device 100 includes a general C-shape formed by the handle 102, the lower support bar 104, and the upper support bar 108. The lower support bar 104 may be secured to the handle 102 at one end 122 with a set screw 124. The lower support bar 104 may alternatively be rigidly and/or permanently coupled with the handle 102 at end 122, according to embodiments of the present invention. The lower support bar 104 may be threadably coupled at its other end 120 to a screw element 106. Screw element 106 may include a handle portion 114 configured to facilitate turning of screw element 106 within lower support bar 104. Screw element 106 may also include an endcap 116 configured for placement against an underside of the calcaneus. The endcap 116 may include nubs or cleats 118 to improve interaction with the calcaneus and minimize slippage of the screw element 106 with respect to the calcaneus, according to embodiments of the present invention. According to some embodiments of the present invention, the screw element 106 includes a threaded outer surface, and the end 120 of lower support bar 104 includes a threaded inner surface around the screw element 106, such that turning the screw element 106 (with handle 114, for example) advances the screw element 106 into or out of the lower support bar 104.

According to some embodiments of the present invention, the handle 102 is rigidly coupled with the upper support bar 108 at joint 132; according to other embodiments of the present invention, the handle 102 is movably and/or adjustably coupled with the upper support bar 108 at joint 132. Upper support bar 108 includes a talus fork 110 and an alignment track 112, according to embodiments of the present invention. The talus fork 110 includes two tines configured for placement at the top of the talus, according to embodiments of the present invention. The talus fork 110 includes a general V-shape and/or U-shape to permit placement over the talus while minimizing tissue damage and potential slippage with respect to the talus, according to embodiments of the present invention. The talus fork 110 may further include nubs or cleats 130 on its underside for improved interaction with the talus and/or to minimize slippage of the talus fork 110 with respect to the talus, according to embodiments of the present invention.

The alignment track 112 is rigidly fixed to the upper support bar 108 and includes one or more slots 126 and/or holes 128 to receive corresponding screws, pegs, and/or protrusions of an alignment device, according to embodiments of the present invention. The alignment device is shown as generally semi-circular in shape, although based on the disclosure provided herein, one of ordinary skill in the art will appreciate that numerous shapes are possible. For example, the alignment track 112 may include a rectangular shape with slots 126 that extend at least partially along the radius of a circle.

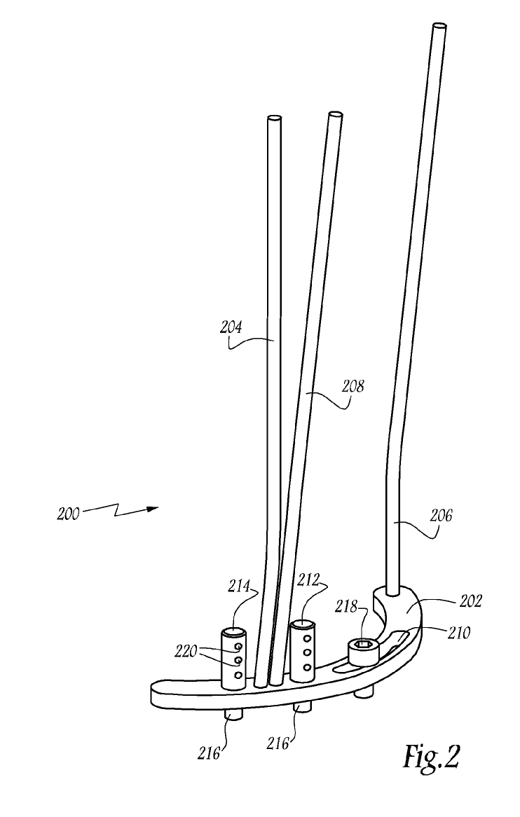

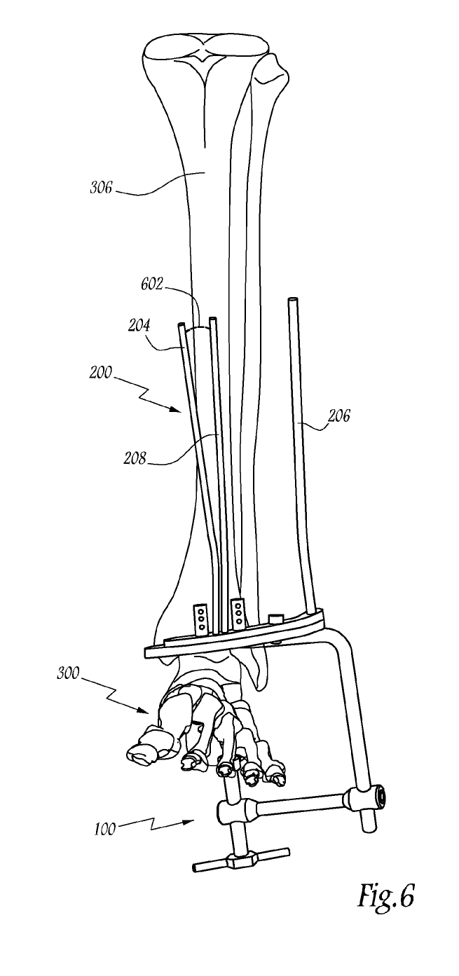

FIG. 2 illustrates a front perspective view of an alignment device 200, according to embodiments of the present invention. Alignment device 200 includes a base 202, alignment rods 204, 206, 208, pin mounts 212, 214, and screw 218, according to embodiments of the present invention. Rods 204, 206, and/or 208 may each also be referred to as angle indicators, according to embodiments of the present invention. Rods 204 and 208 are anterior rods that form an angle with respect to each other, while rod 206 is a lateral rod, according to embodiments of the present invention. The angle between rod 204 and rod 208 may be imparted by bending rod 204 and/or rod 208 at or near the base 202. Alternatively, rods 204 and 208 may be straight or substantially straight rods which are mounted on the base 202 at an angle with respect to each other. Alternatively, rods 204, 208 may be a single rod mounted to the base 202 which is split into two rod halves that form an angle with respect to each other. FIG. 6 illustrates an angle 602 between rod 204 and rod 208 substantially in the coronal plane, according to embodiments of the present invention. Angle 602 may be six degrees, according to embodiments of the present invention. Alternatively, angle 602 may be any desired angle corresponding to a preferred arthrodesis angle or a preferred drilling angle or another preferred angle for the foot with respect to the tibia for a surgical step, according to embodiments of the present invention.

Pin mounts 212, 214 may protrude from a top surface of the base 202, and may include pegs 216 which extend from the bottom surface of the base 202. Pegs 216 may be configured to fit within and interact with the one or more slots 126 of the clamping device 100, according to embodiments of the present invention. Pin mounts 212, 214 may each include fixation pin holes 220 through which one or more fixation pins may be placed for fixation to the tibia. Multiple fixation pin holes 220 may be provided at different vertical levels on pin mounts 212, 214 in order to provide different placement options for the fixation pins, and/or placement options for multiple fixation pins through each pin mount 212, 214, according to embodiments of the present invention.

A screw 218 may also extend through a slot 210 in the base 202; screw 218 may be configured for threadable engagement with the one or more holes 128 in the clamping device 100. One or more compression screws 218 may be included to removably couple the alignment device 200 with the clamping device 100, according to embodiments of the present invention. When screw 218 is placed through slot 210 and engaged with hole 128 and tightened, alignment device 200 does not rotate or translate with respect to clamping device 100, according to embodiments of the present invention. Based on the disclosure provided herein, one of ordinary skill in the art will realize that one or more screws 218 may be used on the base 202 and/or the alignment track 112, and may be used to interface with one or more holes 128 and/or slots 210, 126, and/or one or more nuts and/or washers (not shown) to couple the alignment device 200 with the clamping device 100. The alignment device 200 may also be adjusted relative to and affixed to the clamping device 100 in other ways.

The clamping device 100 may be used for either left side or right side application (and may thus be used on either side of either foot), according to embodiments of the present invention. The alignment device 200 may include different left and right designs for use depending upon which foot and also which side of the foot on which the clamping device 100 is used, such that the anterior alignment rods extend in front of the tibia and the lateral alignment rod extends to the side of the tibia, according to embodiments of the present invention.

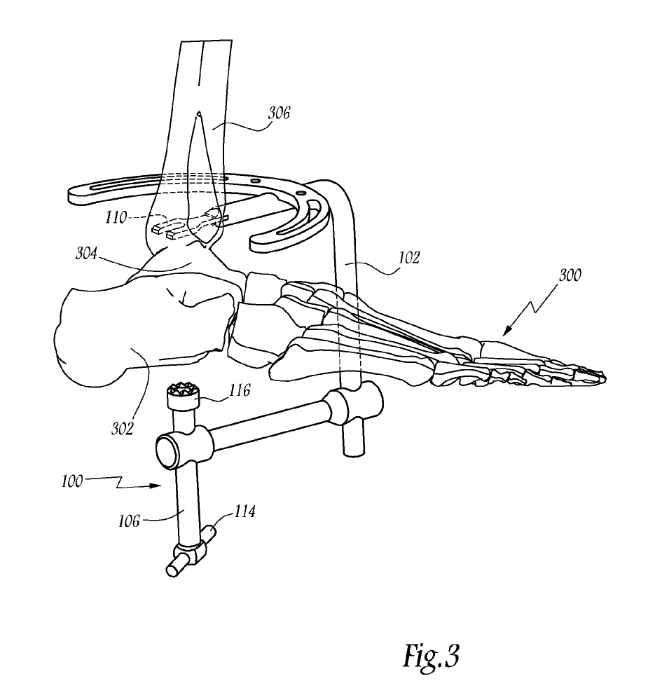

FIG. 3 illustrates a front perspective view of the clamping device 100 being applied to a foot 300, according to embodiments of the present invention. The foot 300 includes a calcaneus 302 and a talus 304. The talus fork 110 is placed over the talus 304 (e.g. over the talus dome and at least partially below the tibia 306), and the screw element 106 is placed below the calcaneus 302. The handle 114 of the screw element 106 is turned to move the endcap 116 closer toward the fork 110 until the endcap 116 contacts the bottom of the calcaneus 302, according to embodiments of the present invention. In external applications, the screw element 106 is turned to move the endcap 116 closer to toward the fork 110 until the endcap 116 contacts the bottom of the subject's foot directly under the calcaneus (e.g. the subject's skin under the calcaneus 302). The screw element 106 may be tightened as desired by the surgeon. For example, the screw element 106 may be tightened to create sufficient compression between the talus fork 110 and the screw element 106 such that adjusting the angle of the foot with respect to the tibia 306 using handle does not result in any significant slippage of the talus fork 110 with respect to the talus 304 or of the endcap 116 with respect to the calcaneus 302. According to some embodiments of the present invention, the endcap 116 is free to rotate with respect to the screw element 106, such that rotating the screw element 106 does not force rotation of the endcap 116, in order to prevent or minimize soft tissue damage.

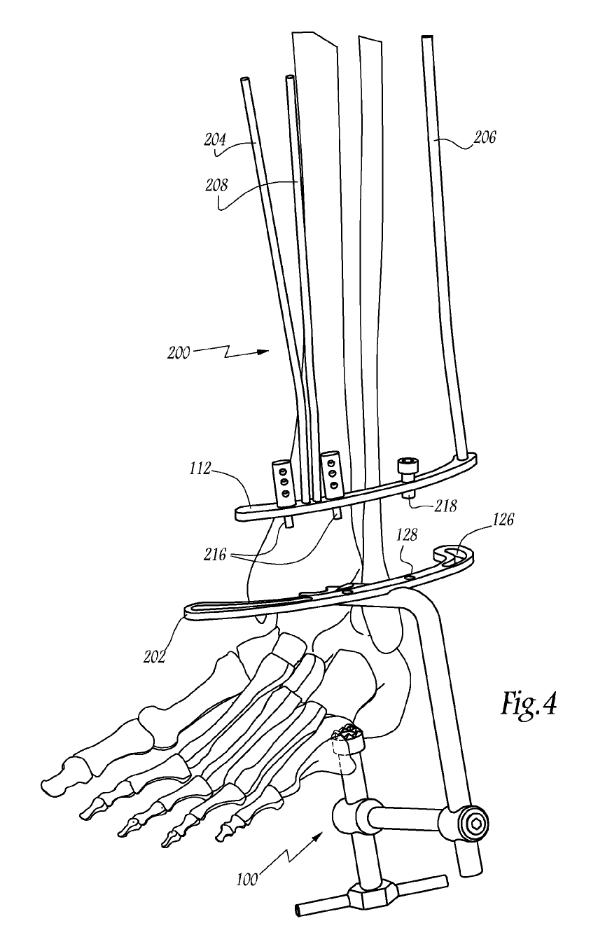

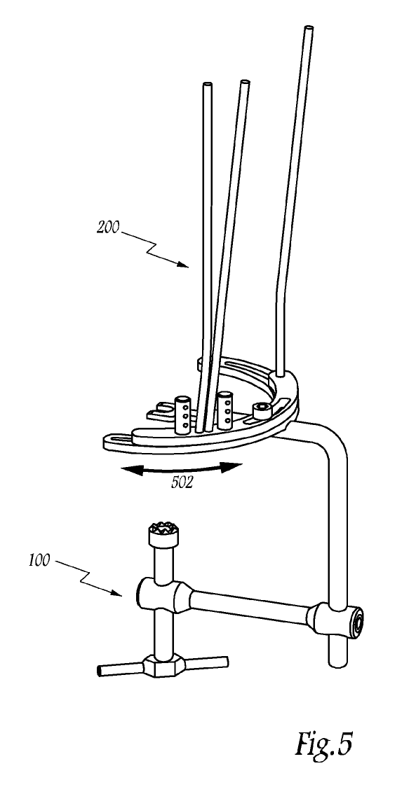

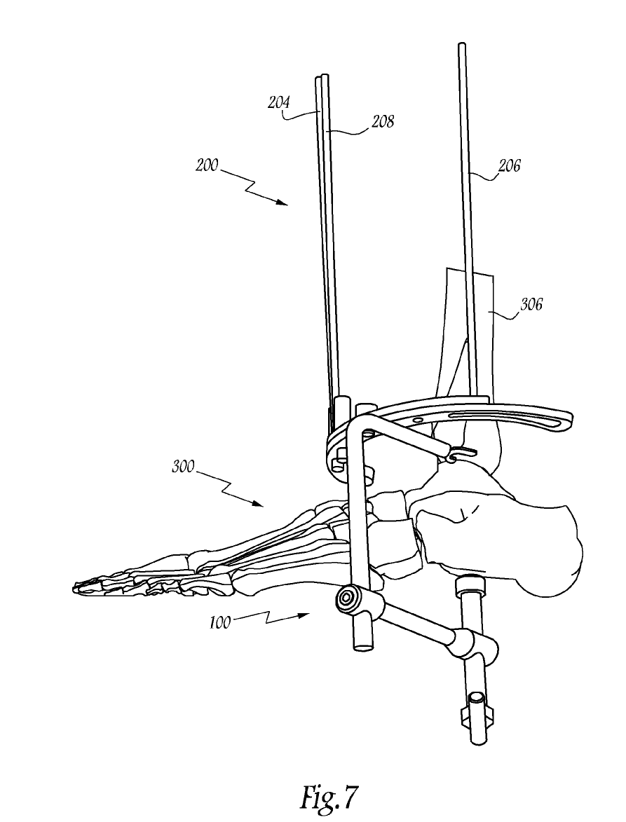

FIGS. 4 and 5 illustrate the alignment device 200 being applied to or mounted to the clamping device 100, according to embodiments of the present invention. Pegs 216 and screw 218 are configured to interface with slots 126 and holes 128, such that pegs 216 slide within slots 126 to permit the base 202 to slide with respect to the alignment track 112 through different radial angular orientations, as illustrated by arrow 502, according to embodiments of the present invention. Once the clamping device 100 has been secured around the talus 304 and calcaneus 302, the foot 300 may be oriented in a desired or preferred arthrodesis position with respect to the tibia 306. While the foot 300 is oriented in the arthrodesis position, the anterior rod 208 of the alignment device 200 may be aligned with the mechanical axis of the tibia 306 in a substantially coronal plane (as illustrated in FIG. 6), and the lateral rod 206 may be aligned with the mechanical axis of the tibia 306 in a substantially sagittal plane (as illustrated in FIG. 7). Once the foot 300 is in the arthrodesis position, and the anterior rod 208 and lateral rod 206 aligned with the mechanical axis of the tibia 306, the alignment device 200 may be coupled with the clamping device 100 such that the position and/or angular orientation of the alignment device 200 is no longer permitted to change with respect to the clamping device 100. This may be accomplished by, for example, tightening one or more compression screws 218 through slot 210 and into one or more holes 128. This step may be referred to as “locking” the alignment device 200 with respect to the clamping device 100.

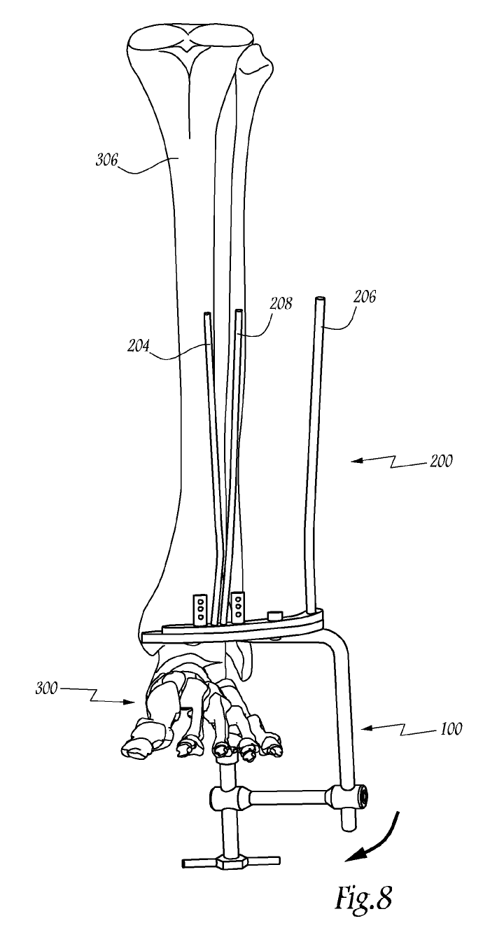

The arthrodesis position of the foot 300 with respect to the tibia 306 is typically at a valgus angle, such as a valgus angle of six degrees. Because a hole for an arthrodesis nail must typically be drilled in a straight line through the foot 300 and into the tibia 306, the foot 300 must often be repositioned from the arthrodesis position into a drilling position. Once the alignment device 200 has been locked to the clamping device 100 as described above, the foot 300 may be moved from the arthrodesis position to the drilling position by aligning the anterior rod 204 with the mechanical axis of the tibia 306 in a substantially coronal plane, as illustrated in FIG. 8, and by aligning the lateral rod 206 with the mechanical axis of the tibia 306 in a substantially sagittal plane, similar to the illustration in FIG. 7, according to embodiments of the present invention. As seen in FIG. 6, the angle through which the foot 300 is rotated between the arthrodesis and drilling positions substantially corresponds to the angle 602 between anterior rods 204 and 208. Thus, the angle 602 may be customized for a particular surgeon and/or a particular operation, arthrodesis nail, or patient. According to some embodiments of the present invention, multiple alignment devices 200 are provided, each having a different angle 602, and each being capable of being locked to the clamping device 100.

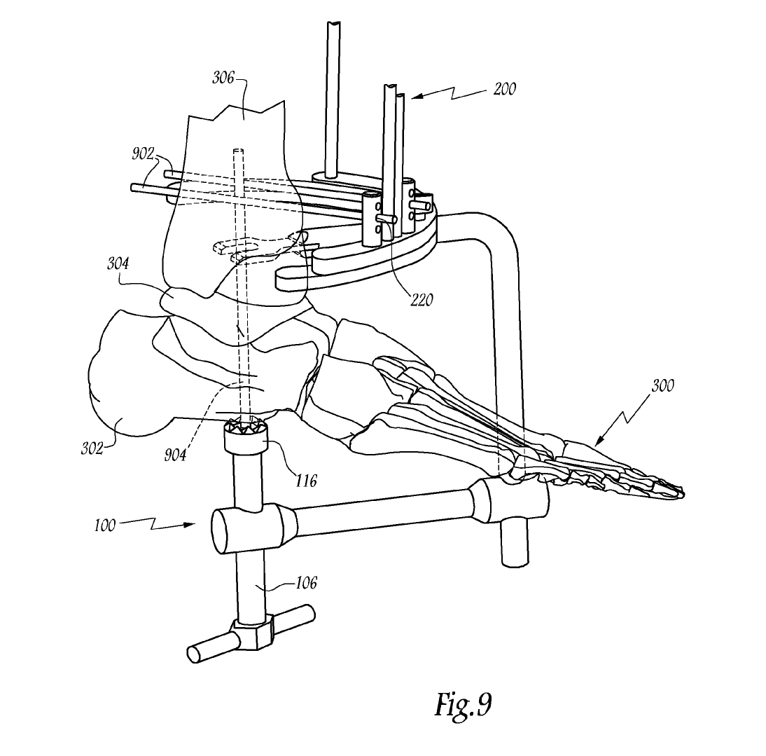

Once the foot 300 is in the drilling position, it may be fixed in the drilling position by placing one or more fixation pins 902 inserted through the one or more fixation pin holes 220 and into the tibia, and/or by placing a long fixation pin 904 through the screw element 106 and into the calcaneus, talus, and tibia, as illustrated in FIG. 9, according to embodiments of the present invention. The screw element 106 may be cannulated in order to permit insertion of long fixation pin 904 and/or insertion of a drill bit therethrough, according to embodiments of the present invention. According to some embodiments of the present invention, the holes for the fixation pins 902, 904 are drilled. According to some embodiments of the present invention, the fixation pin 904 itself is a drill bit. Once the long fixation pin 904 has been placed as illustrated in FIG. 9, the screw element 106 and endcap 116 may be loosened and removed, and another instrument (not shown) may be used to bore around the fixation pin 904, according to embodiments of the present invention. Once the necessary drilling and other preparations have been made for the arthrodesis nail, the arthrodesis nail may be installed.

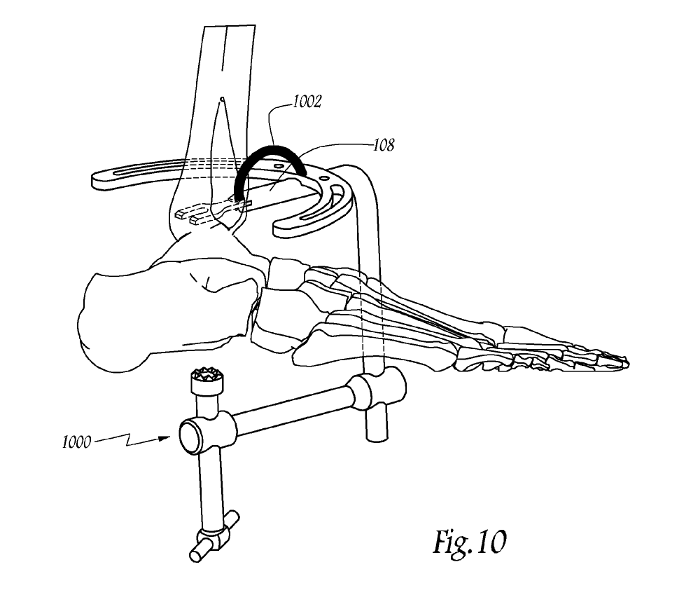

FIG. 10 illustrates a front perspective view of an alternative embodiment of a clamping device 1000, which is similar to clamping device 100 in which the upper support bar 108 is replaced with upper support bar 1002, according to embodiments of the present invention. Providing a curvature to upper support bar 1002 may help minimize soft tissue damage in installation of the clamping device 1000, according to embodiments of the present invention.

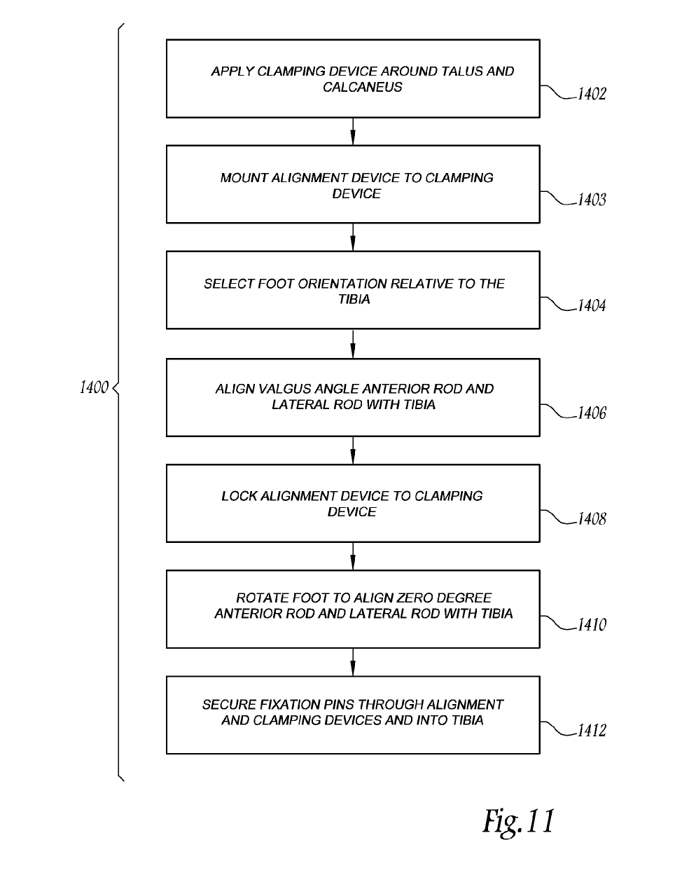

FIG. 11 depicts a flow chart illustrating a method 1400 for using an alignment device and clamping device, according to embodiments of the present invention. A clamping device 100 is applied around a talus 304 and calcaneus 302 (block 1402), as illustrated in FIG. 3. The alignment device 200 is mounted to the clamping device (block 1403) as illustrated in FIGS. 4 and 5. A foot 300 orientation, such as an arthrodesis position of foot 300 with respect to tibia 306, is selected (block 1404). This arthrodesis foot orientation may be at a six degree valgus angle, according to embodiments of the present invention. The valgus angle anterior rod 208 of the alignment device 200 is aligned with the mechanical axis of the tibia 306, and the lateral rod 206 of the alignment device 200 is aligned with the mechanical axis of the tibia 306 (block 1406), as illustrated in FIGS. 6 and 7. The alignment device 200 is locked with respect to the clamping device (block 1408). The foot 300 may be rotated from the selected arthrodesis position to a drilling or other surgical position by aligning the zero degree anterior rod 204 with the mechanical axis of the tibia 306 and the lateral rod 206 with the mechanical axis of the tibia 306 (block 1310), as illustrated in FIG. 8, and securing fixation pins 902, 904 through the alignment device 200 and clamping device 100 and into the tibia 306 (block 1312), as illustrated in FIG. 9.

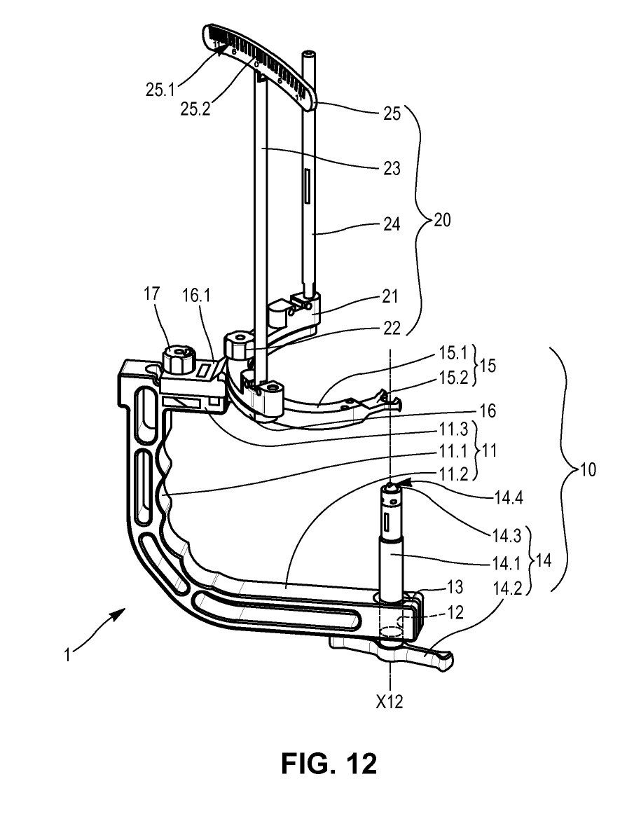

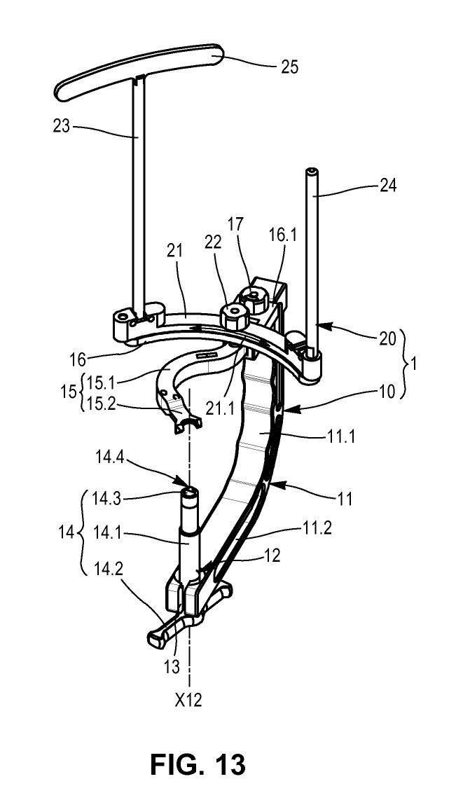

FIGS. 12 and 13 illustrate an alternative positioning guide 1, according to embodiments of the present invention. Positioning guide 1 includes a clamping device 10 and an alignment device 20, according to embodiments of the present invention. Clamping device 10 includes a handle portion 11, which includes a vertical portion 11.1, a horizontal portion 11.2, and an alignment interface portion 11.3. The horizontal portion 11.2 includes a hole 12 configured to receive a locking screw assembly 14, according to embodiments of the present invention. The locking screw assembly 14 includes a screw element 14.1, a handle 14.2, one or more cleats 14.3, and an annular opening 14.4, according to embodiments of the present invention. A slot 13 may be formed in the end of the handle 11, for example at the end of the horizontal portion 11.2, to connect that end with the hole 12, according to embodiments of the present invention.

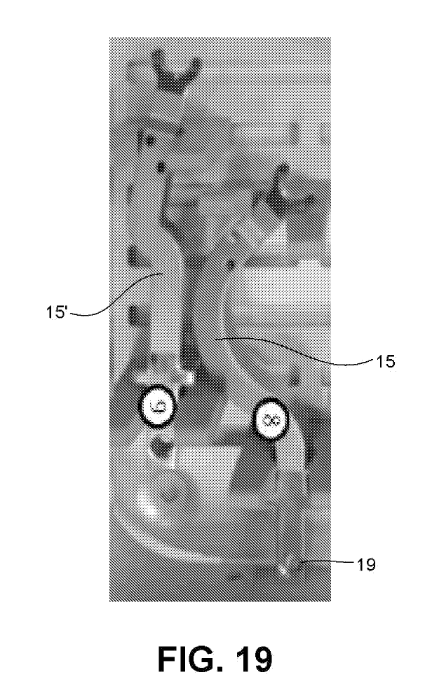

The alignment device 20 is removably attached to the clamping device 10 via bracket 16.1 and knob 17; knob 17 may be turned to tighten or untighten an attached threaded element into or out of a receiving hole in the clamping device 10. The alignment device 20 may be removably attached to the clamping device 10 in other ways; for example, the clamping device 10 may include the knob 17 and/or screw, and the alignment device 10 may include a threaded receiving hole, or other interlocking mechanisms may be used, according to embodiments of the present invention. The upper support bar 15 may be releasable from both the alignment interface portion 11.3 and the bracket 16.1; for example, the upper support bar 15 may be received by one or more slots in the alignment interface portion 11.3 and held between the alignment interface portion 11.3 and the bracket 16.1, according to embodiments of the present invention. The upper support bar may also have a hole 19 formed in its end (see FIG. 19) through which the screw of knob 17 may also pass, to further secure the bracket 16.1, alignment interface portion 11.3, and the upper support bar 15 together, according to embodiments of the present invention. The upper support bar 15 includes a neck portion 15.1 and a fork 15.2, according to embodiments of the present invention. This arrangement also permits different upper support bars 15 to be used interchangeably with the same handle 11 and alignment device 20, according to embodiments of the present invention. For example, as illustrated in FIG. 19, one upper support bar 15′ may be configured for a 10° angle, and another upper support bar 15 may be configured for a 45° angle, according to embodiments of the present invention. The different support bars may have a neck portion 15.1 with a different shape or angular approach. The upper support bar 15 shown in FIG. 12 is configured for a 45° superior approach, meaning that the fork 15.2 extends from the joint at an angle of 45° from the sagittal plane of the foot, according to embodiments of the present invention. The upper support bar 15 is also configured to be used with either a left or right ankle, according to embodiments of the present invention.

The alignment device 20 further includes an alignment track 16 coupled to the bracket 16.1, and a base 21 which is configured to slide along the alignment track 16 as indicated by arrow 21.2 (see FIG. 13) before being releasably secured to the base 21 with locking device 22, according to embodiments of the present invention. The alignment device 20 further includes an alignment rod 24, a protractor stem 23, and a protractor 25 mounted on the protractor stem 23, according to embodiments of the present invention. The protractor may have angular reference marks 25.1, including an index angular reference mark 25.2, according to embodiments of the present invention.

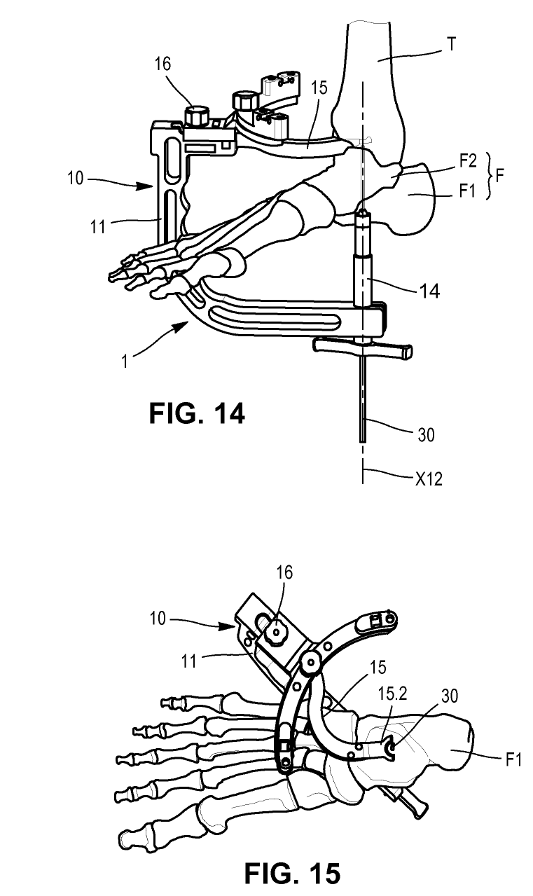

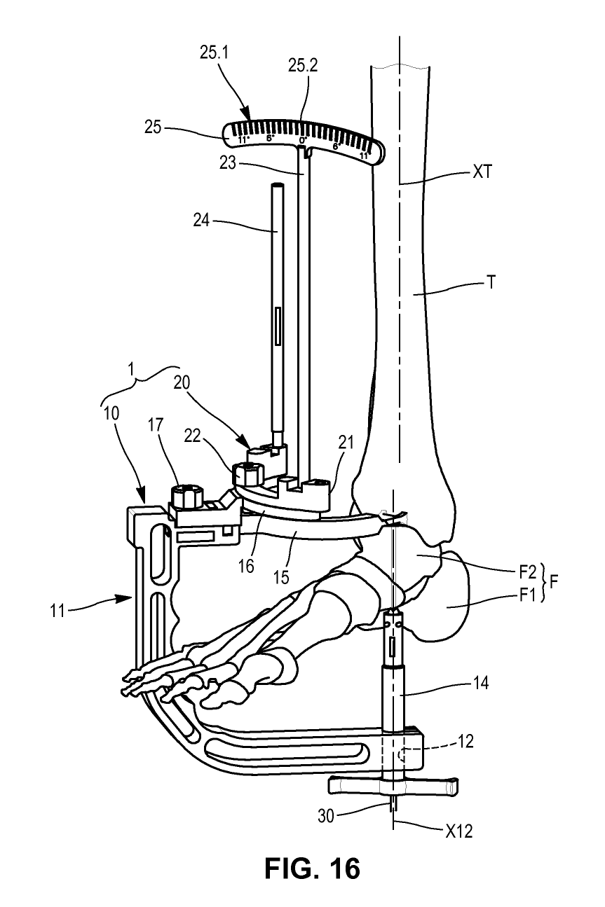

FIGS. 14 to 18 illustrate placement of the positioning guide 1 onto the foot F, according to embodiments of the present invention. The foot F includes a calcaneus F1 and a talus F2, according to embodiments of the present invention. A guide pin 30 may be inserted into the calcaneus F1, for example approximately one centimeter, according to embodiments of the present invention. An upper support bar 15 may be selected, for example an upper support bar 15 having a neck portion 15.1 of a particular shape and/or angular approach, and a particular fork 15.2. For example, one upper support bar may be configured with a 10° angle for a lateral approach, and another may be configured with a 45° angle for an anterior approach, according to embodiments of the present invention. Once the particular upper support bar 15 is selected, it may be attached to the alignment interface portion 11.3 and the bracket 16.1 with knob 17, which acts to reversibly lock the interface portion 11.3, the bracket 16.1, and the upper support bar 15 together, according to embodiments of the present invention. Then, the positioning guide 1 may be positioned in order to center the fork 15.2 on a center of the talar dome of the talus F2, according to embodiments of the present invention.

The guide pin 30 may be inserted into the slot 13 and thus into hole 12, according to embodiments of the present invention. The locking screw assembly 14 may then be inserted over the guide pin 30, by inserting the guide pin 30 through opening 14.4 and then inserting the screw element 14.1 through hole 12, according to embodiments of the present invention. The outside of the screw element 14.1 may include threads, and the inside of the hole 12 may include complementary threads, such that the handle 14.2 of the locking screw assembly 14 may be turned in order to compress the talus F2 against the calcaneus F1 between the cleats 14.3 and the fork 15.2, according to embodiments of the present invention. The opening 14.4 which extends through locking screw assembly 14 may define an implantation axis X12 which is substantially straight and which, when the locking screw assembly 14 is positioned within hole 12, extends between the tines of the fork 15.2, according to embodiments of the present invention. According to embodiments of the present invention, the proximal tip with the cleats 14.3 is free to rotate about the implantation axis X12 with respect to the screw element 14.1, in order to minimize tissue damage at the interface of the locking screw assembly 14 with the calcaneus F1 as the handle 14.2 is turned, according to embodiments of the present invention.

Once the locking screw assembly 14 is tightened in order to compress the calcaneus F1 against the talus F2, the guide pin 30 may be inserted through the calcaneus F1 and the talus F2, and the surgeon may check the exit point of the guide pin 30 to verify that it exits the talar dome of the talus F2 at the fork 15.2, as illustrated in FIG. 15. In other words, the surgeon verifies that the guide pin 30 has substantially followed the implantation axis X12 through the calcaneus F1 and the talus F2, according to embodiments of the present invention. FIG. 15 shows that the proximal end of the guide pin 30 may intersect the talar dome of the talus F1 at a point between the tines of the fork 15.2, according to embodiments of the present invention.

The base 21 of the alignment device 20 may include receptacles configured to releasably accept the protractor stem 23 and the alignment rod 24, according to embodiments of the present invention. The distal ends of the stem 23 and rod 24 may include flat portions to ensure that they are aligned correctly with respect to the base 21. The base 21 may be slid along the alignment track 16 until the desired angular position of the protractor 25 with respect to the tibia T is achieved, and before the base 21 is reversibly locked to the alignment track 16 using locking device 22, according to embodiments of the present invention. For example, the surgeon may desire to place the protractor 25 in a coronal plane (which may also be a plane parallel with the coronal plane), such that the protractor 25 will indicate a rotation angle with respect to the tibia T when the ankle F is rotated laterally or medially. According to embodiments of the present invention, the protractor stem 23 and/or the alignment rod 24 extend from base 21 along longitudinal axes which are substantially parallel to the implantation axis X12, according to embodiments of the present invention. The base 21 is configured to slide with respect to alignment track 16 (for example, along a slot in the alignment track 16) in a manner which maintains the protractor stem 23 and/or alignment rod 24 equidistant from the implantation axis X12. As such, the surgeon may select the desired plane in which measurement of ankle F rotation is desired, by sliding the base 21 along the alignment track 16 and then securing the selected position of the base 21 with respect to the alignment track 16 using locking device 22, according to embodiments of the present invention. For example, the surgeon may adjust the position of the base 21 with respect to the alignment track 16 in order to measure the ankle F rotation angle in a plane that extends along an angle with respect to the coronal plane.

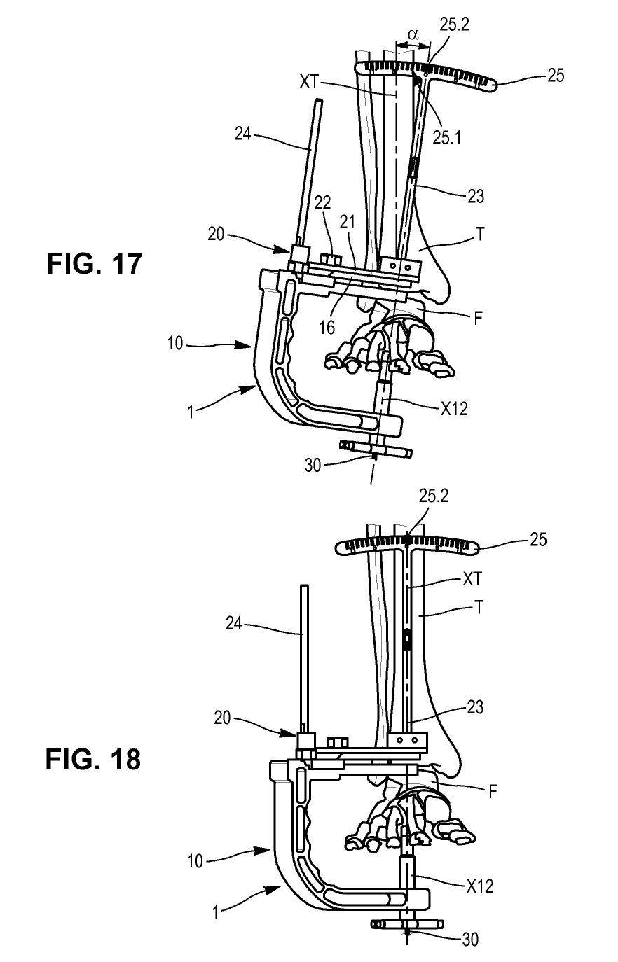

When viewed in an anterior-to-posterior direction, as shown in FIGS. 17 and 18, the foot F may be placed into the desired arthrodesis position, as shown in FIG. 17, according to embodiments of the present invention. Once in the desired arthrodesis position, the surgeon may note the angular reference mark 25.1 which is in alignment with the tibial axis XT; such angular reference mark 25.1 indicates an arthrodesis angle α, according to embodiments of the present invention. For additional precision in measuring the arthrodesis angle α, the alignment rod 24 may optionally also be aligned with the tibial axis XT in the medio-lateral direction (e.g. the direction from left to right in the view of FIG. 17) at the same time that the angle α is noted, according to embodiments of the present invention.

The tibial axis XT may be defined by a longitudinal axis of the tibia T, and may also be referred to as the mechanical axis of the tibia T, according to embodiments of the present invention. If a fixed number of different arthrodesis nails are available to the surgeon, each having a different arthrodesis angle, the protractor 25 face may be configured to display an angular reference mark corresponding to each of the fixed number of different arthrodesis nails, either instead of or in addition to the other angular reference marks 25.1, so that the surgeon can not only measure the arthrodesis angle α, but also determine which available arthrodesis nail is closest in angle to the desired arthrodesis angle α, according to embodiments of the present invention. For example, although the protractor 25 includes angular reference marks 25.1 corresponding to a positive or negative zero to twelve degrees of rotation, the positive and negative six and eleven degree angles are given a visually distinct marking (e.g. calling them out in text or emboldening the selected angles) indicating that the surgeon may choose from two different arthrodesis nails: an arthrodesis nail with a six degree valgus angle, and an arthrodesis nail with an eleven degree valgus angle. In some cases, the maker of the arthrodesis nail may recommend a particular angular range for arthrodesis with which each arthrodesis nail may be used; for example, for the example above in which the surgeon must choose between six degree and eleven degree arthrodesis nails, the six degree nail may be used when α is between two and eight degrees, and the eleven degree nail may be used when α is between nine and fifteen degrees, according to embodiments of the present invention.

Once the angle α is determined, the foot F may be returned from the arthrodesis position of FIG. 17 in which an angle α is formed between the implantation axis X12 and the tibial axis XT, to the implantation position of FIG. 18 in which the implantation axis X12 is substantially aligned with the tibial axis XT, according to embodiments of the present invention. In order to ensure that the implantation axis X12 is aligned with the tibial axis XT, the index reference mark 25.2 of the protractor 25 is aligned with the tibial axis XT while the alignment rod 24 is aligned with the tibial axis XT in the medio-lateral direction (e.g. the direction from left to right in the view of FIG. 18), according to embodiments of the present invention. This aligns the index reference mark 25.2 with the tibial axis XT in one plane (e.g. a coronal plane), and also aligns the alignment rod 24 with the tibial axis XT in another plane (e.g. a sagittal plane) to ensure that the implantation axis X12 intersects the tibia T in a longitudinal sense (e.g. such that the implantation axis X12 is substantially aligned with the tibial axis XT), according to embodiments of the present invention. The index reference mark 25.2 is shown as a zero on protractor 25, although other index reference marks may be used. Also, the protractor stem 23 may be configured to also extend along an angular orientation that corresponds with the index reference mark 25.2, such that the protractor stem 23 itself may act as the index reference mark when aligning the alignment device 20 with the tibial axis XT, according to embodiments of the present invention. Once the implantation position of FIG. 18 is achieved, the guide pin 30 may further be inserted into the tibia T, according to embodiments of the present invention. The locking screw assembly 14 may be unscrewed from the handle 11 and removed from the guide pin 30, and the positioning guide 1 may be removed from the ankle F to permit the subsequent reaming or other preparation of the talus F2, calcaneus F1, and tibia T for implantation of the arthrodesis nail, according to embodiments of the present invention.

The assembly of devices 100 and 200 involves the provision of multiple alignment devices 200, each having a different angle 602, and each being capable of being locked to the clamping device 100, according to embodiments of the present invention. During the selection of the arthrodesis angle 602 with system 100, the surgeon may switch multiple alignment devices 200 onto and off of the clamping device 100 until the desired angle is noted. With positioning guide 1, however, the arthrodesis angle α may be determined without removing or replacing hardware, due to the protractor 25, according to embodiments of the present invention.

The clamping device 10 and/or upper support bar 15 may be configured for use on either left or right ankle. For use on a left ankle, the protractor stem 23 may be inserted into the hole in the base 21 next to where the rod 24 is inserted into base 21 in FIG. 12, and the rod 24 may be inserted into the hole in base 21 next to where the stem 23 is inserted into base 21 in FIG. 12, according to embodiments of the present invention.

According to one alternative embodiment of the present invention (not shown), an alignment rod may extend from alignment device 20 in a direction that is substantially perpendicular to the plane along which the protractor 25 extends. As such, when the protractor 25 is placed into the coronal plane as shown in FIG. 17, such an alignment rod extends orthogonally to the coronal plane; such an alignment rod may be used instead of or in addition to rod 24 in order to ensure that the surgeon is observing the correct angle. For example, the surgeon may visually align such a rod (or its projection in along the tibia T) with the tibial axis XT at the same time as aligning the angular reference mark 25.1, for example when looking toward the bottom of the patient's foot F, in order to ensure that the angle α is measured in the desired plane, according to embodiments of the present invention. Such a rod helps the surgeon ensure that the angle α is not measured while the foot F is overly rotated in one direction or the other about the implantation axis X12, which might result in a distorted observation of angle α, according to embodiments of the present invention.

Although the methods described above mention the use of a guide pin 30, the guide pin 30 is optional in installing the foot positioning guide 1, according to embodiments of the present invention. As such, the clamping device 10 can be positioned about the talus and calcaneus, as described above, before any insertion of a guide pin 30 in the calcaneus, and/or before any insertion of a guide pin into the calcaneus and the talus, according to embodiments of the present invention.

Although rods and protractor markings have been described herein as angle indicators, one of ordinary skill in the art, based on the disclosure provided herein, will recognize that other angle indicators may be used, including but not limited to optical indicators, laser indicators, slots, notches, digital or numeric readouts, and/or the like, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A positioning guide for ankle arthrodesis with respect to a foot and a tibia, the tibia having a tibial axis, the positioning guide comprising:
a clamping device configured to be fixedly held to the foot, the clamping device defining an implantation axis through the foot;
an alignment device configured to be removably coupled with the clamping device, the alignment device comprising a first angle indicator and a second angle indicator,
wherein, when the alignment device is coupled to the clamping device,
the first angle indicator is configured to visually indicate a substantial alignment of the implantation axis with the tibial axis in at least one plane when the first angle indicator is aligned with the tibial axis in the at least one plane and the foot is placed in a first position, and
the second angle indicator is configured to visually indicate an arthrodesis angle formed between the implantation axis and the tibial axis in the at least one plane when the second angle indicator is aligned with the tibial axis in the at least one plane and the foot is placed in a second position.

2. The positioning guide of claim 1, wherein the foot comprises a talus and a calcaneus, wherein the clamping device comprises a talus engagement member configured for placement on a top of a dome of the talus and a calcaneus engagement member configured for placement on a bottom of the calcaneus, and wherein the calcaneus engagement member is configured to be tightened to compress the talus and the calcaneus to fixedly hold the clamping device to the talus and the calcaneus.

3. The positioning guide of claim 2, wherein the calcaneus engagement member is a locking screw assembly.

4. The positioning guide of claim 2, wherein the clamping device comprises a handle, wherein the handle comprises:
a hole positioned along the implantation axis and configured to receive the calcaneus engagement member; and
a lateral slot formed between the hole and an exterior of the handle, the lateral slot configured to receive an orthopedic guide pin to permit sliding of the orthopedic guide pin from the exterior to within the hole.

5. The positioning guide of claim 2, wherein the clamping device further comprises a first upper support bar, the first supper support bar comprising the talus engagement member, the positioning guide further comprising a second upper support bar with a different shape than the first upper support bar, wherein the first and second upper support bars are interchangeable with respect to the clamping device to permit the clamping device to be applied to the foot about different angular orientations with respect to the implantation axis.

6. The positioning guide of claim 3, wherein the locking screw assembly comprises an annular opening that defines the implantation axis.

7. The positioning guide of claim 6, wherein the implantation axis passes through the talus engagement member.

8. The positioning guide of claim 2, wherein the talus engagement member is a talus fork.

9. The positioning guide of claim 1, wherein the alignment device further comprises a third angle indicator, wherein, when the alignment device is coupled to the clamping device, the third angle indicator is configured for visual alignment with the tibial axis both when the foot is in the first position and when the foot is in the second position.

10. The positioning guide of claim 1, wherein the at least one plane is a coronal plane.

11. The positioning guide of claim 9, wherein the at least one plane is a coronal plane, and wherein at least a portion of the third angle indicator is configured to remain in the coronal plane when the foot is in the first and second positions.

12. The positioning guide of claim 9, wherein the at least one plane is a first plane, wherein the third angle indicator extends substantially along a second plane that is orthogonal to the first plane and includes the implantation axis.

13. The positioning guide of claim 1, wherein the first and second angle indicators are configured such that, when the clamping device is fixedly held to the foot and the alignment device is coupled to the clamping device, the first and second angle indicators are positioned anterior to the tibia.

14. The positioning guide of claim 1, wherein the first and second angle indicators are rotatable about the implantation axis with respect to the clamping device before the alignment device is removably coupled with the clamping device.

15. The positioning guide of claim 1, wherein the first and second angle indicators are rods.

16. The positioning guide of claim 9, wherein the third angle indicator is a rod.

17. The positioning guide of claim 1, wherein the alignment device comprises a protractor with angle markings, and wherein the angle markings comprise the first and second angle indicators.

18. The positioning guide of claim 17, wherein the alignment device further comprises a base and a protractor rod connecting the protractor with the base, wherein the protractor rod is configured to visually indicate a substantial alignment of the implantation axis with the tibial axis in the at least one plane when the protractor rod is aligned with the tibial axis in the at least one plane and the foot is placed in the first position.

19. The positioning guide of claim 17, wherein the protractor includes the angle markings both laterally and medially of the first angle indicator to permit use of the alignment device with both a left foot arthrodesis and a right foot arthrodesis.

20. A method for measuring an arthrodesis angle with respect to a foot and a tibia, the tibia having a tibial axis, the method comprising:
placing a clamping device fixedly onto a foot, the clamping device defining an implantation axis through the foot;
coupling an alignment device with the clamping device, the alignment device comprising a first angle indicator and a second angle indicator;
placing the foot in a first position in which the first angle indicator is aligned with the tibial axis to visually indicate substantial alignment of the implantation axis with the tibial axis; and
placing the foot in a second position in which the second angle indicator is aligned with the tibial axis to visually indicate an arthrodesis angle formed between the implantation axis and the tibial axis.

21. The method of claim 20, wherein the alignment device further comprises a protractor with angle markings, wherein the angle markings comprise the first angle indicator and the second angle indicator, wherein placing the foot in the first position comprises aligning an index marking of the angle markings with the tibial axis, and wherein placing the foot in the second position comprises placing the foot in a desired arthrodesis position and observing which of the angle markings is aligned with the tibial axis.

22. The method of claim 21, further comprising determining the arthrodesis angle by determining an angular difference between the index marking and the marking aligned with the tibial axis in the second position.

23. The method of claim 20, wherein the alignment device further comprises a third angle indicator, wherein placing the foot in the first position comprises aligning the third angle indicator with the tibial axis, and wherein placing the foot in the second position comprises aligning the third angle indicator with the tibial axis.

24. The method of claim 20, wherein the alignment device further comprises a third angle indicator, wherein placing the foot in the first position comprises aligning the third angle indicator laterally with the tibial axis, and wherein placing the foot in the second position comprises aligning the third angle indicator laterally with the tibial axis.

25. The method of claim 20, wherein the first and second angle indicators are anterior rods, the alignment device further comprising a third angle indicator that is a lateral rod, wherein placing the foot in the first position comprises aligning the lateral rod laterally with the tibial axis, and wherein placing the foot in the second position comprises aligning the lateral rod laterally with the tibial axis.

26. The method of claim 20, further comprising rotating the alignment device about the implantation axis before coupling the alignment device to the clamping device, to position the first and second angle indicators anteriorly and substantially along a coronal plane with respect to the foot.

27. The method of claim 20, wherein the clamping device comprises a talus engagement member and a calcaneus engagement member, and wherein placing the clamping device fixedly onto the foot comprises placing the talus engagement member on a top of a dome of a talus of the foot and placing the calcaneus engagement member on a bottom of a calcaneus of the foot, the method further comprising tightening the either or both of the talus and calcaneus engagement members to compress the calcaneus and the talus to fixedly hold the clamping device to the talus and the calcaneus.

28. The method of claim 27, wherein the calcaneus engagement member is a locking screw assembly.

29. The method of claim 27, wherein the talus engagement member is a talus fork.
